SEQUENCE LISTING (17 sequences)

SEQ ID NO: 1 - DNA, 59 bp, Artificial Sequence
  Primer
acctcatcgattaatacgactcactatagggataaaataagttagtttgtttgggcaac

SEQ ID NO: 2 - DNA, 38 bp, Artificial Sequence
  Primer
gcatcggatccaaagcttttagtttgttgcccaaacaa

SEQ ID NO: 3 - DNA, 19 bp, Artificial Sequence
  Primer
acgaatatctgcatcggcg

SEQ ID NO: 4 - DNA, 17 bp, Artificial Sequence
  Primer
ccatgtgagccgcgctg

SEQ ID NO: 5 - DNA, 25 bp, Artificial Sequence
  Primer
gacgctctagatccccagcttgttg

SEQ ID NO: 6 - DNA, 33 bp, Artificial Sequence
  Primer
tttctgcaggaaatgatgaattcgtttccaccg

SEQ ID NO: 7 - DNA, 22 bp, Artificial Sequence
  Primer
gatcacccattagccagtcgcg

SEQ ID NO: 8 - DNA, 22 bp, Artificial Sequence
  Primer
gcatgctgcagccctcgatccg

SEQ ID NO: 9 - DNA, 22 bp, Artificial Sequence
  Primer
aaatcggatccagggcttcacc

SEQ ID NO: 10 - DNA, 24 bp, Artificial Sequence
  Primer
ggcttcagacacatgttgctcctc

SEQ ID NO: 11 - DNA, 22 bp, Artificial Sequence
  Primer
atggtcgacgcctatgccatgc

SEQ ID NO: 12 - DNA, 26 bp, Artificial Sequence
  Primer
tgagtcgacgactgggagatgaaagc

SEQ ID NO: 13 - DNA, 27 bp, Artificial Sequence
  Primer
tcggatcccagtcgccgactcatatcc

SEQ ID NO: 14 - DNA, 26 bp, Artificial Sequence
  Primer
agactagtgatccgacagacggacac

SEQ ID NO: 15 - DNA, 45 bp, Artificial Sequence
  Primer
agtcccgggaaaaggaggtcactaaatgaacacgattaacatcgc

SEQ ID NO: 16 - DNA, 25 bp, Artificial Sequence
  Primer
gctgtatcgatttggcgttacgcgt

SEQ ID NO: 17 - DNA, 55 bp, Artificial Sequence
  Primer
taatacgactcactatagggataaaataagttagtttgtttgggcaacaaactaa

(19) United States
(12) Patent Application Publication
(10) Pub. No.: US 2004/0072206 A1
(43) Pub. Date: Apr. 15, 2004

(54) Method for identifying modulators of transcription

(76) Inventors: Jeffrey Errington, Oxford (GB); Helena Barbara Thomaides, Oxon (GB)

Correspondence Address: FULBRIGHT & JAWORSKI, LLP, 1301 MCKINNEY, SUITE 5100, HOUSTON, TX, 77010-3095, US

(21) Appl. No.: 10/433,585

(22) PCT Filed: Dec. 4, 2001
(86) PCT No.: PCT/GB01/05354

(30) Foreign Application Priority Data
Dec. 4, 2000 (GB) 0029539.4

(51) Int. Cl. C12Q 1/68

(52) U.S. Cl. 435/6

(57) ABSTRACT

A method for identifying a modulator of RNA polymerase (RNAP) comprises providing a host cell which expresses a first RNAP and has a first and second polynucleotide construct. The first polynucleotide construct comprises a first promoter operably linked to a first gene wherein the first gene is transcribed by the first RNAP. The second polynucleotide construct comprises a second promoter operably linked to a second gene which is a reporter gene and wherein the second reporter gene is transcribed by a second RNAP. The host cell is provided with a source of the second RNAP. A test substance is contacted with the host cell under conditions that would permit the expression of the first and second genes in the absence of the test substance. It can be determined thereby whether the test substance modulates RNAP.

### FIELD OF THE INVENTION

The invention relates to a method for identifying modulators of transcription in a host cell and in particular modulators of RNA polymerase (RNAP). The method can be used to identify inhibitors of RNAP. Such inhibitors can be used to kill organisms or restrict growth. The inhibitors may be used in the treatment of infections in the human or animal body, for example as antibiotics to treat bacterial infection.

### BACKGROUND TO THE INVENTION

RNA polymerase is used by organisms to transcribe DNA or RNA. RNA polymerases of different organisms may exhibit substantial structural differences. For example RNA polymerase of bacteria or other infecting agents may be structurally very different from the RNA polymerase of the cell that they infect. RNA polymerase is thus a target for modulation and particularly for inhibition. It is desirable to identify agents which modulate RNA polymerase which may be useful in altering the growth pattern of a host cell.

Bacterial RNA polymerase (RNAP) is a useful target for antibiotics because of the differences in structure and regulation when compared to RNA polymerase of higher organisms. The activity of a target compound in a host cell, such as a bacterium may be determined using a reporter gene. Generally, transcription of the reporter gene will be induced at the same time as or before administering the target compound. If a target inhibits RNAP, transcription of the reporter gene will be blocked. However, reporter gene expression may also be inhibited through non-specific effects on diverse cell processes such as translation, intermediary metabolism, membrane integrity, etc. Thus, there is a need to establish an assay which can be used to identify specific modulators of a host RNA polymerase.

### SUMMARY OF THE INVENTION

A novel cell-based assay is now provided which can be used to identify a modulator of RNA polymerase. Such an assay is particularly useful for identifying an inhibitor of RNA polymerase which does not inhibit a second RNA polymerase such as heterologous RNA polymerase. Such an inhibitor may be used to restrict growth of an organism, for example as an antibiotic for treating bacterial infection.

The invention provides a method for identifying a modulator of RNA polymerase (RNAP) comprising:

providing a host cell which expresses a first RNA polymerase and having a first polynucleotide construct comprising a first promoter operably linked to a first gene, wherein the first gene is transcribed by the first RNAP; a second polynucleotide construct comprising a second promoter operably linked to a second gene which is a reporter gene, wherein the reporter gene is transcribed by a second RNAP; and a source of the second RNAP;

contacting a test substance with the host cell under conditions that would permit the expression and activity of the first and second genes in the absence of the test substance;

and determining thereby whether the said substance modulates RNAP.

In a preferred aspect, the source of the second RNAP comprises a third polynucleotide construct comprising a third promoter operably linked to a gene encoding the second RNAP. The assay is carried out under conditions which allow expression of the second RNAP within the host cells. One or more of the promoters may be inducible promoters. In one preferred aspect, the first promoter and the second promoter are inducible in response to the same stimulus. The third promoter may comprise a constitutive or inducible promoter. In an alternative preferred aspect, the assay is carried out in the absence of inducer.

Preferably, the method is used to determine whether a test substance modulates host RNAP activity but does not modulate the second RNAP activity. Preferably, a test substance which is identified inhibits bacterial RNAP but which does not inhibit a heterologous RNAP. A modulator or an inhibitor of RNAP may be used in the treatment of the human or animal body. Preferably, an inhibitor is identified and may be used in the treatment of bacterial infection. The invention also relates to pharmaceutical compositions comprising an inhibitor and a pharmaceutically acceptable carrier.

### BRIEF DESCRIPTION OF THE FIGURES

FIG. 1A is a schematic representation of an assay of the invention.

FIG. 1B is a scheme representing the assay system for inhibitors of bacterial RNA polymerase.

FIG. 2 is a schematic representation of the genetic organisation of strain PL37. P_{xyl}-lacZ is placed in the yybCB locus. The rpoT7 gene is under the control of the IPTG-inducible P_{spac} promoter, at the ywhED locus. The control reporter is located at the amyE locus.

FIG. 3 shows the DNA sequence upstream of the gus gene comprising the P_{T7X}-gus fusion.

FIG. 4 shows the effect of IPTG concentration on expression of the control (P_{T7X}-gus; A) and test (P_{xyl}-lacZ; B) reporters of the assay strain PL37. Strain PL37 (diamonds) was grown in the presence of different concentrations of IPTG and assayed for β-glucuronidase (A) and β-galactosidase (B) activity. Strains PL9 (triangles) and PL34 (squares), strains that do not possess the rpoT7 gene, were also examined under the same conditions as negative controls.

FIGS. 5 to 7 show the effects of specific and non-specific antibiotics on the expression of the control (P_{T7X}-gus; FIG. 5) and test (P_{xyl}-lacZ; FIG. 6) reporters of the assay strain PL37. PL37 was grown in the presence of different concentrations of antibiotics, and a no compound control (DMSO) and assayed for β-glucuronidase and β-galactosidase activity (see also data in Table 2). Data is also shown as the ratio of β-glucuronidase to β-galactosidase activity (FIG. 7).

### DETAILED DESCRIPTION OF THE INVENTION

The present invention provides a method for identifying a modulator of RNA polymerase (RNAP). In particular, the method can be used to identify a modulator of RNAP which does not modulate a second RNAP. The method is a cell-based assay. Preferably, the method is used to investigate modulation of the host cell RNA polymerase.

The host cell may comprise a prokaryotic or eukaryotic cell, such a bacterium, yeast cell or mammalian host cell. Preferably, the RNA polymerase to be investigated comprises a bacterial RNAP. The second RNAP may comprise a heterologous RNAP derived from a different organism. For example, where the host cell is a bacterial cell, the second RNAP may be of viral or eukaryotic origin. In an alternative embodiment, the second RNAP comprises a RNAP endogenous to the host cell. For example, the method may be used to look for modulators of different RNAP of bacterial cells. For example, the method can be used to investigate modulators of RNAP σ^{D} and σ^{A} of B. subtilis. Alternatively the host cell is a eukaryotic cell provided with a source of bacterial RNAP to identify inhibitors of bacterial RNAP which do not affect the host cell RNAP.

The host cell is provided with a first polynucleotide construct comprising a first promoter operably linked to a first gene. The first promoter is recognised by the first RNAP such that the first gene is transcribed by the first RNAP. The host cell additionally comprises a second polynucleotide construct comprising a second promoter operably linked to a second gene namely a reporter gene wherein the second reporter gene is recognised by the second RNAP and the second gene is transcribed by the second RNAP. The assay is carried out under conditions such that the first RNAP does not transcribe the second gene and the second RNAP does not transcribe the first gene. In addition the first gene and second gene are selected so that it is possible to differentiate between expression of the first gene and expression of the second gene or expression of both genes.

In one aspect of the invention, the first gene comprises a first reporter gene and the second gene comprises a second reporter gene. The reporter genes encode products which can readily be detected. For example, the reporter product may be detectable by fluorescent, luminescent or other standard reporting techniques. The reporter gene products may comprise an enzyme such as β-galactosidase, production of which may be identified by use of a colourigenic or fluorogenic enzyme substrate. Other reporter genes include β-glucuronidase, green fluorescent protein (GFP) and variants thereof, luciferase, chloramphenicol acetyltransferase, catechol oxidase, an antigen which may readily be recognised by an antibody, other, affinity ligands such as streptavidin/biotin or protein A which may be detected by antibodies etc. The first and second reporter genes are selected such that it is possible to differentiate between expression of the first reporter gene and expression of the second reporter gene.

In such an assay of the present invention, expression of both the first and second reporter gene should occur in the absence of an inhibitor. If an inhibitor is then applied which acts to prevent the expression of one of the reporter genes, the signal detected from the other reporter gene may be amplified due to competition for cellular components. This effect will make even a small difference in the expression, of the reporter genes easier to detect.

In an alternative aspect of the invention, the first gene encodes a repressor, and in particular an unstable repressor which, when expressed, prevents transcription of the second reporter gene. In this embodiment, transcription of the first gene leads to expression of the repressor and thus prevents expression of the second reporter gene. However, if expression of the first gene is inhibited, no repressor will be made and/or existing repressor will decay. Transcription of the second reporter gene may then occur. If the substance under test inhibits the first RNAP in a non-specific manner, expression of the second reporter gene will also be inhibited either through an inhibition of the second RNAP or by inhibition of other cellular processes such as translation. However, if the second reporter gene is expressed, the substance under test will comprise a specific inhibitor of the first RNAP.

Examples of repressors include Xyl R, Tet R, Lac I, cI of phage lambda. Preferably, the repressor is unstable such that transcription of the second RNAP will only be inhibited if repressor continues to be expressed from the first gene.

As outlined above, the first RNAP or RNAP subunit is generally expressed by the host cell. In some embodiments, it may be desirable to assay a first RNAP which is not endogenous to the host cell. In this embodiment a source of the first, RNAP would also be supplied to the host cell. The second RNAP may be a heterologous RNAP and needs to be supplied to the host cell. Examples of suitable heterologous RNAP include the RNAP of phage T7 or T3. These RNAP's are small single subunit enzymes and thus are easier to supply to the host cell than the less preferred eukaryotic or bacterial RNAP's which comprise a number of subunits.

Heterologous RNAP may be provided as a protein. For example, RNAP may be packaged in virus particles. A host cell such as a bacterial host cell may be infected with phage containing or producing RNAP just prior to or at the same time as addition of the test compound such that the host cell is provided with a source of a second RNAP.

In an alternative embodiment, the second RNAP is endogenous to the host cell and the first RNAP is supplied to the cell. For example the assay could be carried out in a eukaryotic cell to look for inhibitors of bacterial RNA polymerase. Bacterial RNAP is supplied to the cell.

In a preferred embodiment, an RNAP which is not endogenous to the host cell is provided as a third polynucleotide construct comprising a third promoter operably linked to a gene encoding the RNAP. The third polynucleotide construct is provided such that this RNAP will be expressed under the conditions of the assay and preferably will be expressed at some level prior to addition of the test compound. In this way expression of this RNAP is not affected by the conditions of the test and the second gene would be expressed under the conditions of the test in the absence of the test substance. Where the RNAP comprises a number of different subunits, the cell is provided with a polynucleotide construct or constructs for expression of each of the subunits required for RNAP activity.

In an alternative embodiment, the second RNAP does not comprise a heterologous RNAP. For example, bacterial cells express several different RNAP's each made up of five subunits. The σ subunit varies between the different RNAP forms, examples being σ^{A} and σ^{D} in B. subtilis. The second reporter gene is provided with a promoter which is recognised by only one of these RNAP'S, and the first gene is provided with a promoter which is recognised only by another of these RNAPs. This assay may be used to identify modulators which specifically act on only one of the bacterial RNAP's.

Each of the polynucleotide constructs comprises a promoter operably linked to the gene to be expressed. In one aspect, the first and second promoters are inducible promoters. Alternatively, the first and third promoters may be inducible promoters. The conditions required to induce one or more of these promoters may be applied to the host cell when adding the test substance; or before or after adding the test substance, during the course of the assay. Such inducing conditions would allow for the expression of the genes in the absence of the test substance. Examples of inducers of proteins include xylose, tetracycline, lactose and derivatives thereof including IPTG, arabinose and gluconate or a change of temperature. For example, if the promoter is controlled by a temperature-sensitive repressor, the promoter can be induced by increased temperature. Inducible promoters include promoters which are completely inactive in the absence of inducers, leading to no gene expression, and promoters which are inducible, but do not require inducers for gene expression. For example, in the absence of a suitable inducer, a lower level of gene expression may still occur. In an alternative embodiment, the assay may be carried out in the absence of inducer.

The term “operably linked” refers to a juxtaposition wherein the components described are in a relationship permitting them to function in their intended manner. Thus, a regulatory sequence such as a promoter, “operably linked” to a coding sequence is positioned in such a way that expression of the coding sequence is achieved under conditions compatible with the regulatory sequence.

In the embodiment of the invention where the first gene encodes a repressor such as an unstable repressor, the second promoter is selected to be one which is controlled by the repressor. The first gene may be provided with a constitutive promoter or is induced to express the repressor prior to the beginning of the assay such that the repressor is expressed and prevents expression of the second reporter gene prior to commencement of the assay.

Where an RNAP is provided as a polynucleotide construct, it is preferably under the control of a constitutive promoter. If it is under the control of an inducible promoter, the assay is carried out under conditions such that expression of this RNAP will take place in the presence of the test substance. Suitable constitutive promoters are those promoters which are expressed strongly during growth in rich media. Examples of suitable promoters would include ribosomal RNA gene promoters and promoters that are normally subject to regulation but which are relieved of this regulation by mutation or the absence of their regulatory proteins such as P_{R} or P_{L} of phage lambda, in the absence of phage and P_{Lac} in the absence of functional Lac I. Where the second reporter gene has a promoter which is controlled by a repressor encoded by the first gene, the third polynucleotide construct has a promoter which is preferably selected to provide a reasonably low level of expression such that expression of the second reporter gene does not tale place in the presence of the repressor.

The polynucleotide constructs may be provided as vectors for transformation of the host cell. The polynucleotide constructs may be provided on the same or different vectors. Vectors may be used to replicate the vectors in a compatible host cell. The vectors may be for example, plasmid vectors provided with an origin of replication and optionally a regulator of the promoter. The vectors may contain one or more selectable marker genes, for example, ampicillin or chloramphenicol resistance gene for selection in bacterial cells or a G418 or a zeocin resistance gene for selection in mammalian cells.

The invention also relates to a host cell transformed, conjugated or transduced with first and second polynucleotide constructs for the expression of the first and second reporter genes. Optionally, the host cell may also express the second RNAP.

In a preferred aspect of the invention the host cell comprises a bacterial cell, providing a source of bacterial RNAP. Such cells may be useful to identify a modulator of bacterial RNAP and in particular an inhibitor of bacterial RNAP. In view of the similarity in RNAP expressed by different bacterial species, the assay may be carried out in any bacterial cell and will be useful to identify an inhibitor which is expected to inhibit any bacterial RNAP. Alternatively, the assay may be used to establish whether an inhibitor may be identified which affects RNAP from a specific or a number of selected bacterial species but which does not affect other bacterial RNAP's. As outlined above, the method can also be used to identify a specific inhibitor of bacterial RNAP comprising a σ^{A} subunit which does not have any effect on bacterial RNAP comprising another a factor e.g. σ^{D} and vice versa. Preferably, the host cell under investigation is E. coli or B. subtilis. Preferably, the assay is used to identify an inhibitor of bacterial RNAP which inhibits across a broad range of bacteria such as E coli, Salmonella, Bacillus, Streptococcus, Staphylococcus and Meningococcus and thus can be used in the treatment of bacterial infections as a broad spectrum antibiotic. The assay could alternatively be used to identify an antibiotic which acts against specific bacterial species. Alternatively the assay uses a eukaryotic cell supplied with a source of bacterial RNAP to identify inhibitors of bacterial RNAP which do not effect eukaryotic RNAP.

The assay of the invention is used to screen for compounds which modulate RNAP. Any suitable format may be used for the assay for identifying a modulator of RNAP activity. The way in which the assay is carried out will depend in part of the nature of the first and second reporter genes. In some instances, it may be possible to monitor for production of the reporter protein on a single sample. In some instances it may be necessary to divide a sample containing the host cells following administration of the test substance in order to monitor separately for first and second reporter gene activity. The conditions of the assay are selected such that the host cell may grow in the absence of the test substance.

Additional control experiments may be appropriate. The progress of the assay can be followed in the presence and in the absence of the test substance. Known RNAP modulators, such as rifampicin and streptolydigin which are inhibitors of bacterial RNAP, may be used as positive controls in order to show a comparable or similar effect in a test substance.

Suitable test substances which can be tested in the above assays include combinatorial libraries, defined chemical entities, peptide and peptide mimetics, oligonucleotides and natural product libraries, such as display (e.g. phage display libraries) and antibody products.

Test substances may be used in an initial screen of, for example, ten substances per reaction, and the substance of these batches which show inhibition or activation tested individually. Test substances may be used at concentrations from 1 μM to 1000 μM, preferably from 1 μM to 100 μM, more preferably from 1 μM to 10 μM. Complex mixtures of natural origin (e.g. filtrates from bacterial cultures, or plant extracts) may be used.

A substance which inhibits or activates the activity of RNAP may do so by binding the enzyme. Such enzyme inhibition may be reversible or irreversible. An irreversible inhibitor or activator dissociates very slowly from its target enzyme because it becomes very tightly bound to the enzyme (either covalently or non-covalently). Reversible inhibition or activation, in contrast with irreversible inhibition or activation is characterised by a rapid dissociation of the enzyme-inhibitor/activator complex.

The test substance may be a competitive inhibitor. In competitive inhibition, the enzyme can bind substrate (forming an enzyme-substrate complex) or inhibitor (enzyme-inhibitor complex) but not both. Many competitive inhibitors resemble the substrate and bind the active site of the enzyme. The substrate is therefore prevented from binding to the same active site. A competitive inhibitor diminishes the rate of catalysis by reducing the proportion of enzyme molecules bound to a substrate.

The inhibitor may also be a non-competitive inhibitor. In non-competitive inhibition, which is also reversible, the inhibitor and substrate call bind simultaneously to an enzyme molecule. This means that their binding sites do not overlap. A non-competitive inhibitor acts by decreasing the turnover number of an enzyme rather than by diminishing the proportion of enzyme molecules that are bound to substrate.

The inhibitor can also be a mixed inhibitor. Mixed inhibition occurs when an inhibitor both effects the binding of substrate and alters the turnover number of the enzyme.

A substance which inhibits the activity of RNAP may also do so by binding to the substrate. The substance may itself catalyse a reaction of the substrate, so that the substrate is not available to the enzyme. Alternatively the inhibitor may simply prevent the substrate binding to the enzyme.

A substance which is an activator may increase the affinity of the substrate for the enzyme or vice versa. If this is the case the proportion of enzyme molecules bound to substrate molecules is increased and the rate of catalysis will thus increase. An activator may increase the affinity of a substrate for an enzyme by binding to the enzyme or substrate or both.

A modulator of RNAP activity is one which produces a measurable reduction or increase in RNAP activity in the assays described above.

Preferred inhibitors are those which inhibit RNAP activity by at least 10%, at least 20%, at least 30%, at least 40% at least 50%, at least 60%, at least 70%, at least 80%, at least 90%, at least 95% or at least 99% at a concentration of the inhibitor of 1 μg ml⁻¹, 10 μg ml⁻¹, 100 μg ml⁻¹, 500 μg ml⁻¹, 1 mg ml⁻¹, 10 mg ml⁻¹, 100 mg ml⁻¹. Preferably, the assay may identify an inhibitor of RNAP which inhibits at least 80 or 90% activity at a concentration of 10 μg ml⁻¹.

Preferred activators are those which activate bacterial RNAP activity by at least 10%, at least 25%, at least 50%, at least 100%, at least, 200%, at least 500% or at least 1000% at a concentration of the activator 1 μg ml⁻¹, 10 μg ml⁻¹, 100 μg ml⁻¹, 500 μg ml⁻¹, 1 mg ml^{-1,} 10 mg ml⁻¹, 100 mg ml⁻¹. Preferably an activator activates by at least 50% at 10 μg ml⁻¹.

The percentage inhibition or activation represents the percentage decrease or increase in activity of RNAP in a comparison of assays in the presence and absence of the test substance. Any combination of the above mentioned degrees of percentage inhibition or activation and concentration of inhibitor or activator maybe used to define an inhibitor or activator of the invention, with greater inhibition or activation at lower concentrations being preferred.

Therapeutic Uses

Modulators of RNAP and in particular inhibitors of bacterial RNAP activity may be used to restrict the growth of organisms and in particular bacteria. Such inhibitors may be used to treat bacterial conditions in humans or animals and thus may be used as antibiotics to treat such bacterial infection.

Modulators of RNAP activity may be administered in a variety of dosage forms. Thus, they can be administered orally, for example as tablets, troches, lozenges, aqueous or oily suspensions, dispersible powders or granules. The inhibitors may also be administered parenterally, either subcutaneously, intravenously, intramuscularly, intrasternally, transdermally or by infusion techniques. The modulators may also be administered as suppositories. A physician will be able to determine the required route of administration for each particular patient.

The formulation of a modulator for use in prophylaxis or treatment will depend upon factors such as the nature of the exact modulator, whether a pharmaceutical or veterinary use is intended, etc. A modulator may be formulated for simultaneous, separate or sequential use.

A modulator of RNAP activity is typically formulated for administration in the present invention with a pharmaceutically acceptable carrier or diluent. The pharmaceutical carrier or diluent may be, for example, an isotonic solution. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in known manner, for example, by means of mixing, granulating, tabletting, sugar-coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for intravenous or infusions may contain as carrier, for example, sterile water or preferably they may be in the form of sterile, aqueous, isotonic saline solutions.

A therapeutically effective amount of a modulator is administered to a patient. The dose of modulator may be determined according to various parameters, especially according to the substance used; the age, weight and condition of the patient to be treated; the route of administration; and the required regimen. Again, a physician will be able to determine the required route of administration and dosage for any particular patient. A typical daily dose is from about 0.1 to 50 mg per kg, preferably from about 0.1 mg/kg to 10 mg/kg of body weight, according to the activity of the specific inhibitor, the age, weight and conditions of the subject to be treated, the type and severity of the degeneration and the frequency and route of administration. Preferably, daily dosage levels are from 5 mg to 2 g.

The following Examples illustrate the invention:

### EXAMPLE 1

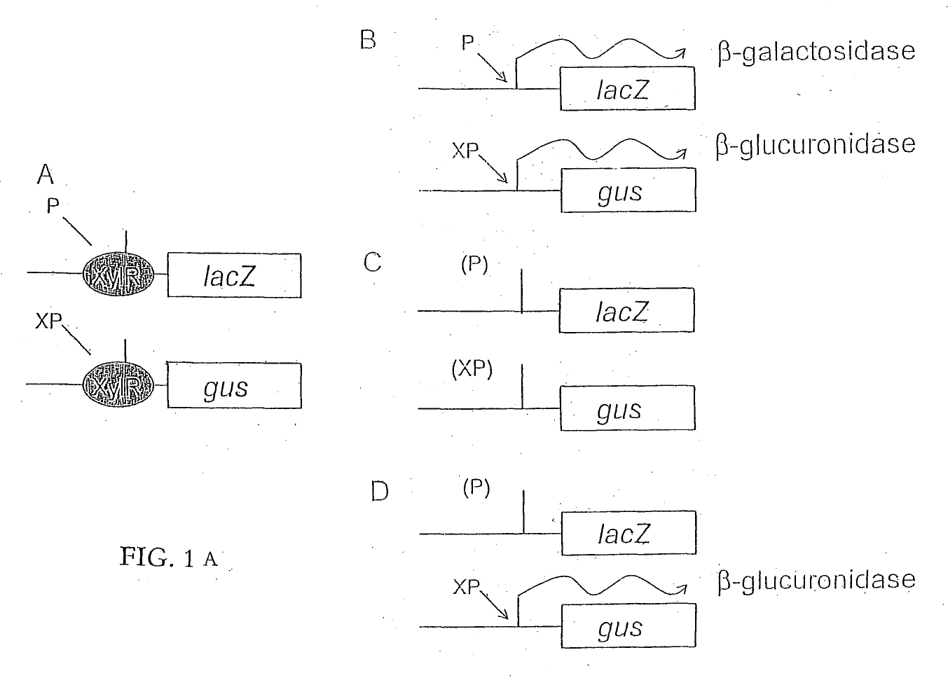

FIG. 1A shows a schematic representation of an embodiment of the invention. The RNA Polymerase inhibitor test strain contains two reporter genes, one encoding the enzyme β-galactosidase; the other encoding β-glucuronidase. In the uninduced state (A) (prior to addition of test chemicals and inducer) the repressor, in this example XylR, binds to the promoters of both genes and prevents transcription from being initiated. The former promoter is otherwise recognisable by host σ^{A} containing RNA polymerase (P), the latter by control RNA polymerase of exogenous origin (XP), for example, phage T7 RNA polymerase, or host RNA polymerase containing a different or exogenous sigma factor (e.g. σ^{D}).

Under induced conditions (presence of the sugar xylose), in the presence of potential chemical inhibitors of RNA polymerase function, three possible outcomes are illustrated (B, C, D). If there is no inhibition, both reporter genes can now be recognised by their respective RNA polymerase forms and both enzymes are made (B). If there is a non-specific inhibitor of RNA polymerase function (C) (parentheses indicate that the RNA polymerase is non-functional or otherwise ineffective), neither gene will be transcribed and neither enzyme will be made (non-specific inhibitors could also abolish formation of both reporter enzymes at a post-transcriptional stage). Finally, in the presence of a specific inhibitor of host RNA polymerase (D), only β-glucuronidase will be made.

The strain X is a derivative of the standard laboratory strain of Bacillus subtilis 168. It has been modified in three ways.

1. It carries a xylose-inducible promoter driving transcription of the well known reporter gene lacZ, encoding the enzyme β-galactosidase. The reporter gene is silent or expressed at low levels in the absence of xylose because the endogenous XylR protein binds to the promoter region, reducing the ability of RNAP to initiate transcription. On addition of xylose or similar sugar, the repressor leaves the promoter allowing RNAP to transcribe the gene strongly, resulting in increased synthesis of β-galactosidase. A simple colourigenic substrate, ONPG was used to detect the formation of the enzyme.

2. Strain X also carries a second reporter gene comprising a promoter recognised by the RNAP of bacteriophage T7. This promoter has been modified so as to be recognised and repressed by the same XylR repressor as controls the first reporter. Consequently, the promoter cannot be utilised by T7 RNAP unless xylose is present. The promoter drives transcription of a second reporter gene, gus, encoding the enzyme β-glucuronidase. Activity of this enzyme was followed in parallel with the β-galactosidase by use of the fluorogenic substrate MUGluc.

3. Finally, strain X carries a copy of the gene encoding T7 RNAP, which is expressed constitutively. Thus, the cells always contain some molecules of T7 RNAP which could initiate transcription at the promoter described in 2 above, provided that repression by the XylR promoter has been relieved by addition of xylose.

The assay was done by growing a culture of strain X in the absence of xylose and then dispensing aliquots of culture into the wells of a microtitre plate. Each well contained xylose to induce expression of the two reporter genes. Some wells additionally contained antibiotics or chemicals of various classes. The plate was incubated for 30 min to allow accumulation of the two reporter enzymes, then the cells were lysed and the two enzyme activities were measured.

Three types of response were seen. In the control wells to which no additional compounds were added, or in wells containing chemicals of a non-toxic nature, reporter activities were unaffected. In wells to which agents affecting aspects of cellular function unrelated to transcription were added, both reporter activities were reduced or abolished. Finally, in wells containing the specific inhibitors of bacterial RNA polymerase, rifampicin and streptolydigin, only the reporter enzyme driven by the T7 RNAP (β-glucuronidase) was active.

### EXAMPLE 2

Experimental Methods

General Methods

DNA manipulations and E. coli transformations were carried out as described previously (Sambrook, J., E. F. Fritsch and Maniatis, T. (1989). Molecular cloning: a laboratory manual. Cold Spring Harbor, N.Y., Cold Spring Harbor Laboratory Press.). All cloning was done in E. coli DH5α (Gibco BRL). Selection of transformants was on Oxoid nutrient agar containing ampicillin (100 μg ml⁻¹). B. subtilis strains were transformed using a modification of the method of Kunst and Rapoport (J. Bacteriol. 177: 2403-2407 (1995); Genes Dev. 12: 3419-3430 (1998)) or of Anagnostopoulos and Spizizen (Anagnostopoulos, C. and Spizizen, J. (1961) J. Bacteriol. 81: 741-746; Jenkinson, H. F. (1983). J. Gen. Microbiol. 129: 1945-1958.). Transformants were selected on Oxoid nutrient agar containing as necessary, chloramphenicol (5 μg ml⁻¹), spectinomycin (75 μg ml⁻¹), kanamycin (5 μg ml⁻¹), tetracycline (10 μg ml⁻¹) and/or IPTG (1 mM). Strains and plasmids used in this study are listed in Table 1 below.

TABLE 1
Strains and plasmids used in this study

| / | | Source/ |
|---|---|---|
| Strain/ | | construc- |
| plasmid | Relevant characteristics | tion |
| B. subtilis | | |
| 168 | trpC2 | Labora- |
| | | tory stock |
| PL9 | trpC2 Ω(amyE::P_{T7X}-gus neo)pHT7 | This study |
| PL13 | trpC2 Ω(yybBC::cat P_{xyl}-lacZ) | This study |
| PL22 | trpC2 Ω(amyE::P_{T7}-gus neo)pHT9 | This study |
| PL27 | trpC2 Ω(vwhED::spc P_{spac}lacI)pHT16 | This study |
| | Ω(amyE::P_{T7}-gus neo)pHT9 | |
| PL28 | trpC2 Ω(ywhED::spc′ tet lacI)pSG1404 | This study |
| | Ω(amyE::P_{T7}-gus neo)pHT9 | |
| PL31 | trpC2 Ω(ywhED::spc P_{spac}-rpoT7 lacI tet) | This study |
| | Ω(amyE::P_{T7}-gus neo)pHT9 | |
| PL33 | trpC2 Ω(ywhED::spc P_{spac}-rpoT7 lacI) | This study |
| | Ω(amyE::P_{T7X}-gus neo)pHT7 | |
| PL37 | trpC2 Ω(ywhED::spc P_{spac}-rpoT7 lacI) | This study |
| | Ω(amyE::P_{T7X}-gus neo)pHT7 | |
| | Ω(yybBC::cat P_{xyl}-lacZ) | |
| PL34 | trpC2 Ω(ywhED::spc P_{spac}-rpoT7 lacI) | This study |
| E. coli | | |
| DH5α | F⁻ endA1 hsdR17 supE44 thi-1 λ⁻ recA1 gyrA96 | Gibco |
| | relA1 Δ(lacZYA-argF)U169 φ80 dlacZ ΔAM15 | BRL |
| BL21 | F⁻ hsdS gal φDE3(P_{lacUV5}-rpoT7) | (1) |
| (DE3) | | |
| Plasmids | | |
| pHT7 | pMLK83 containing P_{T7}xyl operator (P_{T7X}) | This study |
| pHT9 | pMLK83 containing P_{T7} | This study |
| pHT12 | PSG1301 containing spc P_{spac} lacI | This study |
| pHT13 | pHT12 containing the 3′ end of ywhE | This study |
| pHT15 | pSG1403 containing the 3′ end of ywhD | This study |
| pHT16 | pHT13 containing the 3′ end of ywhD | This study |
| pMLK83 | bla amyE3′ gus neo amyE5′ | (2) |
| pMUTIN4 | bla P_{spac} lacZ lacI ermC | (3) |
| pSG1301 | bla cat | (4) |
| pSG1403 | bla spc P_{spac} lacI | (5) |
| pSG1404 | bla spc′ tet lacI | (5) |
| pRD96 | bla cat P_{xyl} | (6) |
| pET3a | bla P_{T7}φ_{T7}t | (1) |

Construction of a P_{T7X}-gus Fusion

Two overlapping oligonucleotide primers, F 1(5′-ACCTCATCGATTAATACGACTCACTATAGGGATAAAATAAGTTAGTTTGTTTGGGCAAC-3′) and R2 (5′-GCATCGGATCCAAAGCTTTTAGTTTGTTGCCCAAACAA-3′) were used as templates in an end-filling/amplification reaction with Taq DNA polymerase. The ˜100 bp fragment amplified contained the T7 promoter separated from the xylose operator sequence by 9 bp (Gartner et al (1992). Mol. Gen. Genet. 232: 415-422.). This fragment was digested with ClaI and BamHI, and then ligated to appropriately digested pMLK83, to generate plasmid pHT7. The sequence upstream of the gis gene was sequenced from a PCR product, amplified using primers, GUS-R3 (5′-ACGAATATCTGCATCGGCG-3′) and 9661H (5′-CCATGTGAGCCGCGCTG-3′), and pHT7 as the template. Primer 9661H was used for sequencing carried out by the Sequencing Service of the Sir William Dunn School of Pathology, University of Oxford. This plasmid was transformed into strain 168 with selection for kanamycin resistance. A strain (PL9) was selected on its amylase deficient phenotype, confirming that the construct had been inserted by a double-crossover event at the amyE locus.

Construction of a P_{T7}-gus Fusion

The phage T7 promoter (PT7) was excised from plasmid pET3a as a SalI-BamHI fragment, gel-purified and ligated to SalI- and BamHI-digested pMLK83, to generate pHT9. This plasmid was transformed into strain 168 with selection for kanamycin resistance. A strain (PL22) was selected on its amylase deficient phenotype, confirming that the construct had been inserted by a double-crossover event at the amyE locus.

Construction of a Strain Harbouring a P_{xyl}-lacZ Fusion in the yybCB Intergenic Region

The lacZ gene from plasmid pMUTIN4 was amplified by PCR to include the spoVG ribosome binding site using primers lacZ-fw1 (5′-GACGCTCTAGATCCCCAGCTTGTTG-3′) and R-lacZ1 (5′-TTTCTGCAGGAAATGATGAATTCGTTTCCACCG-3′), introducing XbaI and PstI sites, respectively. A fragment upstream of the yybCB intergenic region was amplified by PCR, using primers yybE-F (5′-GATCACCCATTAGCCAGTCGCG-3′) and yybC-R (5′-GCATGCTGCAGCCCTCGATCCG-3′), introducing a PstI site at the 3′ end of the fragment. Similarly a fragment downstream of the yybCB intergenic region was amplified by PCR using primers yybB-F (5′-AAATCGGATCCAGGGCTTCACC-3′) and yyat-R (5′-GGCTTCAGACACATGTTGCTCCTC-3′), introducing a BamHI site at the 5′ end of the fragment. The cat P_{xyl} fragment was excised from plasmid pRD96 as a BamHI-XbaI fragment and gel-purified. A ligation of the cat P_{xyl} fragment, the BamHI-digested downstream yybCB fragment; the XbaI-PstI-digested lacZ fragment and the PstI-digested upstream yybCB fragment was transformed into 168 to allow the insertion of cat P_{xyl}-lacZ into the yybBC locus. A chloramphenicol-resistant strain (PL13) was isolated that stained blue on agar plates containing 5-bromo-4-chloro-3-indolyl-β-D-galactopyranoside (X-gal; 100 μg ml⁻¹), indicating the activity of β-galactosidase.

Construction of a Strain Carrying a Spectinomycin Resistance Determinant in the ywhED Intergenic Region

The spc P_{spac} lacI fragment was excised from plasmid pSG301 by KpnI-SacI digestion, gel-purified and ligated to KpnI- and SacI-digested pSG1403 to give plasmid pHT12. An 820 bp fragment from the ywhE gene, covering part of the ywhE-ywhD intergenic region, was amplified by PCR using primers YWHE.FW (5′-ATGGTCGACGCCTATGCCATGC-3′) and YWHE.RV (5′-TGAGTCGACGACTGGGAGATGAAAGC-3′). The fragment was digested with SalI and ligated to SalI-digested and phosphatase-treated pHT12 to give plasmid pHT13. A 640 bp fragment from the ywhD gene, covering part of the ywhE-ywhD intergenic region, was amplified by PCR using primers YWHD.FW (5′-TCGGATCCCAGTCGCCGACTCATATCC-3′) and YWHD.RV (5′-AGACTAGTGATCCGACAGACGGACAC-3′). The fragment was digested with SpeI and BamHI and ligated to SpeI-BamHI digested pHT13 to create pHT16. This plasmid was transformed into strain PL22 with selection for spectinomycin resistance and chloramphenicol sensitivity, which is indicative of integration of spc P_{spac} lacI by a double crossover at the ywhED intergenic region. This created strain PL27.

Construction of a Strain Expressing rpoT7 Inserted into the ywhED Intergenic Region

Plasmid pSG1403 was cut with SpeI and BamHI and ligated to appropriately cut ywhD fragment, amplified as described above, to give plasmid pHT15. Strain PL27 was transformed with plasmid pSG1404 with selection for tetracycline resistance and spectinomycin sensitivity. This created strain PL28 with which plasmid pHT15 has ample regions of homology (the spc and lacI ywhD fragments) for cloning at the ywhD locus, rpoT7, encoding T7 RNAP, was PCR-amplified from Escherichia coli BL21(DE3) using primers T7F3 (5′-AGTCCCGGGAAAAGGAGGTCACTAAATGAACACGATTAACATCGC-3′) and T7R3 (5′-GCTGTATCGATTTGGCGTTACGCGT-3′), thereby introducing a B. subtilis ribosome binding site upstream of the 7rpoT7 gene. The rpoT7 fragment was digested with SmaI and ClaI and ligated to SmaI-ClaI cut pHT15. The ligation mix was transformed directly into PL28, with selection for spectinomycin resistance and an ability to express β-glucuronidase [i.e. to fluoresce in the presence of 4-methylumbelliferyl-β-D-glucoronide (50 μg ml⁻¹)]. This created strain PL31. Strain 168 was then transformed with chromosomal DNA from strain PL31 with selection for spectinomycin resistance and tetracycline sensitivity to create strain PL34, containing P_{spac}-rpoT7 stably integrated at the ywhED locus.

Construction of an Assay Strain Containing a Functional Dual Reporter System

Strain PL34 was transformed with plasmid pHT7, containing the P_{T7X}-gus fusion, with selection for kanamycin resistance and an amylase-deficient phenotype. This created strain PL33, which contained a P_{T7X}-gus fusion inserted by double crossover at the amyE locus. PL33 also harboured the rpoT7 gene which is transcribed by the host RNAP in an IPTG-dependent manner. The test reporter (P_{xyl}-lacZ), recognised by the host RNAP was produced to strain PL33 by transformation with PL13 chromosomal DNA, with selection for chloramphenicol resistance, to give the assay strain PL37.

T7 RNAP Induction Experiments

Cultures of strains PL9, PL34 and PL37 were grown overnight in PAB. To induce expression of rpoT7, each culture was split into aliquots that were supplemented with different concentrations of IPTG (0; 0.01; 0.05, 0.1; 0.5 mM final) and incubated at 37° C. for 1 hour. At this stage cultures were diluted down in unsupplemented PAB medium. 20 μl samples of each culture were added to the wells of microtitre plates containing 20 μl of 2% v/v DMSO. Plates were incubated at 37° C. for two hours and assay mix was added to detect reporter enzyme activity (see below).

The Effect of Xylose on the Reporter System of PL37

Strain PL37 was grown and treated as described above. 20 μl samples of each culture were added to the wells of microtitre plates containing 20 μl of 2% v/v DMSO supplemented with different concentrations of xylose (0; 0.025; 0.05; 0.1; 0.2; 0.4% w/v). Plates were incubated at 37° C. for 2 hours and assay mix was, added as described below.

Testing PL37 Against an Antibiotic Panel

20 known antibiotics were tested at a range of concentrations to determine their effect on test and control reporters within the assay. The following antibiotics were tested at concentrations ranging from 128 μg/ml to 0.125 μg/ml: carbenicillin, lincomycin, novobiocin, trimethoprim lactate, chloramphenicol, ofloxacin, monensin, polymyxin, kanamycin, streptolydigin, oxolinic acid, nalidixic acid, spectinomycin and bacitracin. Erythromycin, cephalexin, penicillin G, ampicillin and vancomycin were tested at concentrations ranging from 16 μg/ml to 0.016 μg/ml, rifampicin was tested at 0.5 μg/ml to 0.0005 μg/ml to accommodate its low MIC value. Cultures of PL37 grown in PAB, were diluted to 0.05 A₆₀₀ and then grown for 2 hours at 37° C. in PAB supplemented with 0.02 mM IPTG. Cells were pelleted and frozen. They were later resuspended and diluted in PAB supplemented with 0.02 mM IPTG and grown at 37° C. to early exponential phase. Cultures were then diluted to 0.05 A₆₀₀ in PAB and added in 20 μl volumes to wells of microtitre plates containing 20 μl volumes of the antibiotics. Plates were incubated at 37° C. for two hours, and assay mix was added to detect reporter enzyme activity (see below).

Detection of β-galactosidase and βglucuronidase Activities

Following the addition of 160 μl of assay mix (1.3×Z buffer containing 94 μg/ml lysozyme, 8.4 μg/ml 4-methylumbelliferyl-β-D-galactoside, 0.53 μg/ml resorufin-β-D-glucuronide and 0.05% Triton) to each well, the microtitre plate was incubated in the dark for 30 mini or 1 hour at room temperature. Fluorescence was read on a BMG FLUOstar Galaxy at excitation/emission 355/460 nm and 544/590 nm, respectively, with gains set at 5 and 25, respectively.

Results

Construction of the Assay Strain PL37

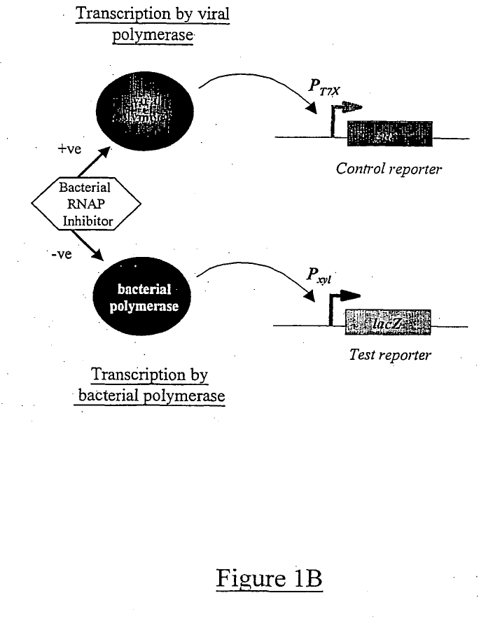

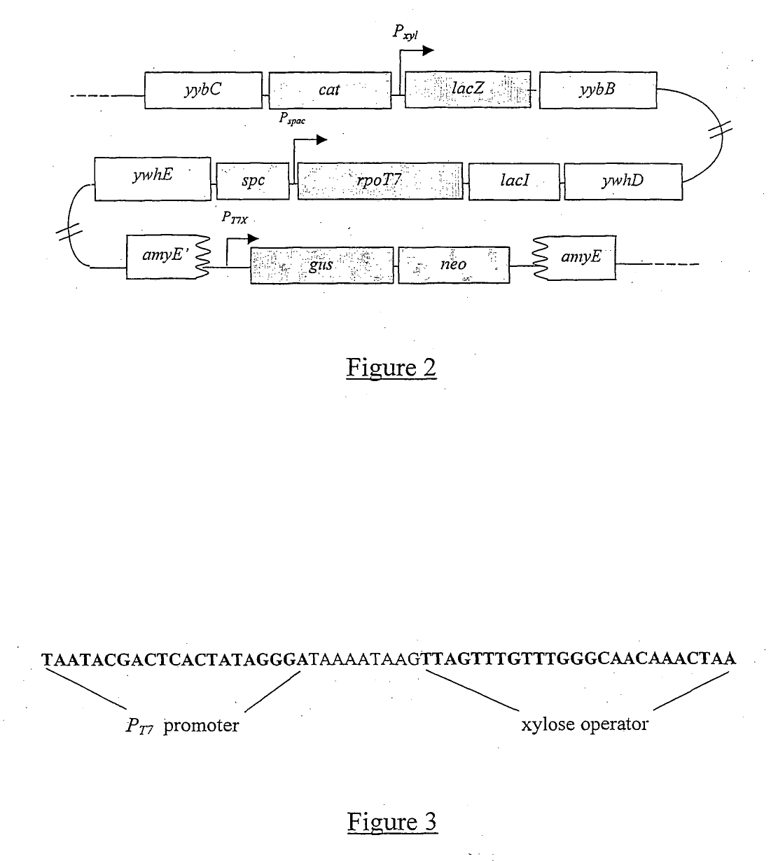

Strain PL37 (FIG. 2) contains the test reporter, xylose-inducible P_{xyl}-lacZ, and a copy of rpoT7, encoding T7 RNAP, which is induced by IPTG (P_{spac}-rpoT7). The control reporter comprises the P_{T7} promoter separated by 9 bp from the xylose operator sequence, upstream of the gus gene (FIG. 3). For reasons that are not clear, this promoter was not subjected to repression by XylR, but this did not affect the outcome of the assay.

Expression of the Control Reporter is IPTG-Dependent

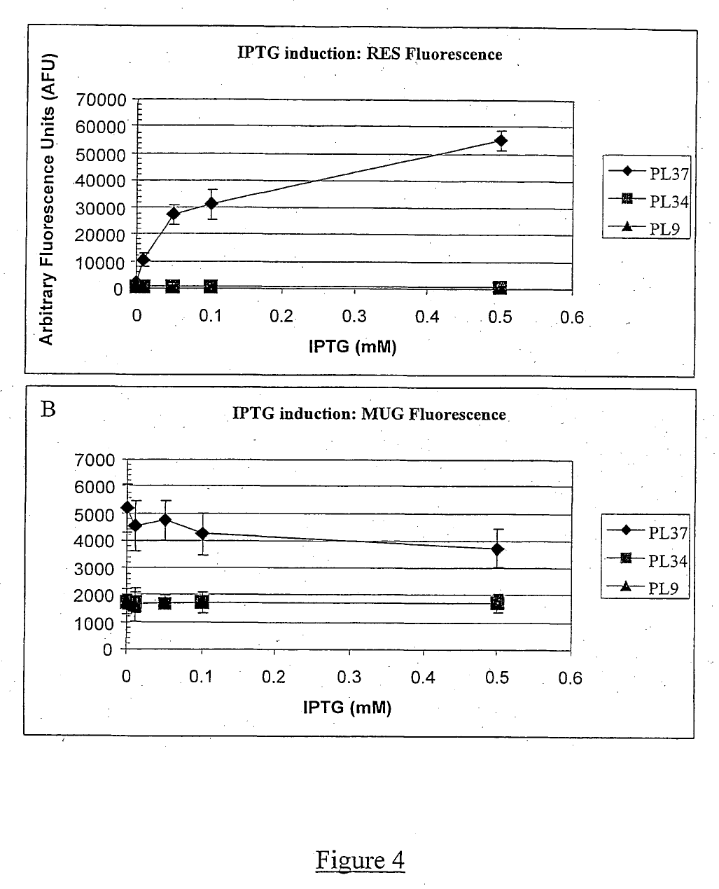

PL37 was grown in the presence of different concentrations of IPTG, which should induce expression of the rpoT7 gene, encoding the phage T7 RNAP that in turn would transcribe the control reporter gene, gus. As shown in FIG. 4A, the activity of the control reporter was IPTG-dependent. In contrast, expression of the control reporter from the parental strain lacking the P_{spac}-rpoT7 construct (PL9) was at the same background levels as a strain containing this construct but lacking a gus reporter (PL34). Consequently, strain PL37 showed gus reporter expression that was dependent on induction of rpoT7 gene expression by IPTG and hence transcribed by the T7 RNAP. In addition, as shown in FIG. 4B, the test reporter of strain PL37 was expressed in the absence of IPTG and remained unchanged when IPTG was added.

Effects of Antibiotics on the Assay

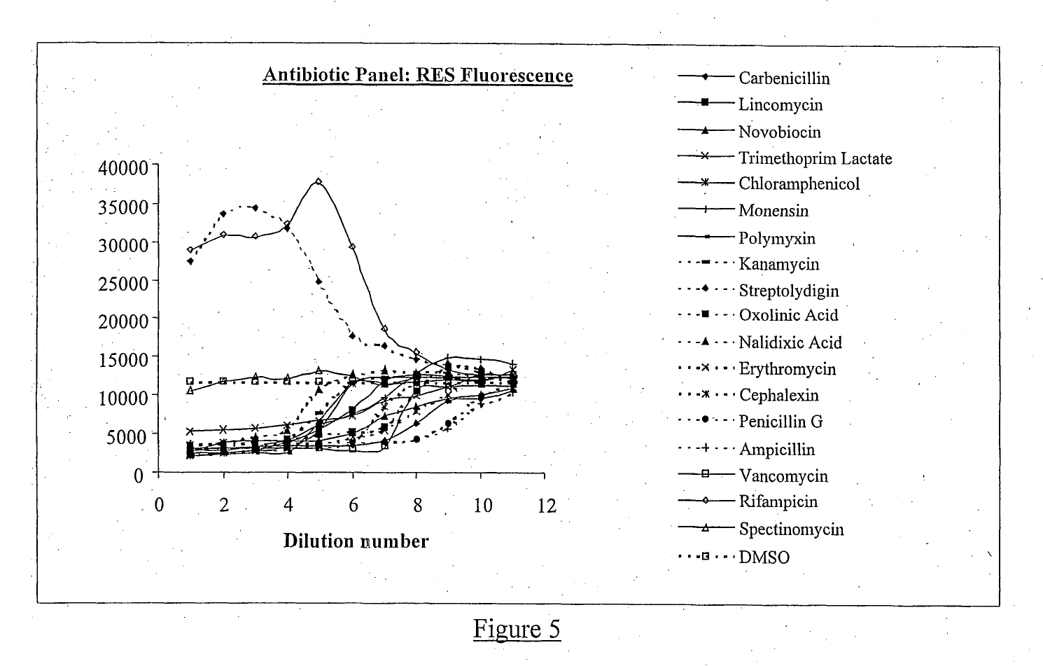

FIGS. 5 to 7 show fluorescence data and the ratio of control:test reporter (RES:MUG) data obtained from an experiment testing a selection of antibiotics, at a range of concentrations, against assay strain PL37. Dilution 1 was the highest concentration tested and dilution 11 was the lowest concentration tested. Table 2 lists individual values for the RES:MUG ratio under the same conditions.

TABLE 2
Ratio values of the control reporter (RES): test reporter (MUG) at various
antibiotic concentrations

| | Dilution Number | | | | | | | | | | |
|---|---|---|---|---|---|---|---|---|---|---|---|
| Antibiotic | 1 | 2 | 3 | 4 | 5 | 6 | 7 | 8 | 9 | 10 | 11 |
| Carbenicillin | 1.37 | 1.49 | 1.59 | 1.63 | 1.66 | 1.64 | 1.60 | 1.03 | 1.00 | 1.05 | 1.07 |
| Lincomycin | 1.09 | 1.20 | 1.29 | 1.61 | 1.91 | 1.40 | 1.08 | 0.99 | 1.02 | 1.03 | 1.08 |
| Novobiocin | 1.33 | 1.70 | 1.66 | 1.49 | 1.24 | 0.81 | 0.85 | 1.05 | 1.14 | 1.18 | 1.19 |
| Trimethoprim Lactate | 0.62 | 0.55 | 0.54 | 0.52 | 0.54 | 0.58 | 0.68 | 0.65 | 0.63 | 0.81 | 1.00 |
| Chloramphenicol | 1.10 | 1.22 | 1.27 | 1.40 | 1.90 | 1.51 | 1.20 | 1.16 | 1.14 | 1.13 | 1.17 |
| Ofloxacin | 0.06 | 0.19 | 0.42 | 0.80 | 1.21 | 1.72 | 1.72 | 1.29 | 1.54 | 1.29 | 0.99 |
| Monensin | 1.74 | 1.42 | 1.45 | 1.79 | 2.06 | 2.00 | 1.88 | 1.74 | 1.54 | 1.37 | 1.33 |
| Polymyxin | 1.19 | 1.22 | 1.21 | 1.23 | 1.44 | 1.24 | 1.20 | 1.15 | 1.16 | 1.16 | 1.17 |
| Kanamycin | 1.07 | 1.18 | 1.50 | 1.92 | 2.15 | 1.24 | 1.17 | 1.16 | 1.13 | 1.11 | 1.18 |
| Streptolydigin | 16.44 | 16.68 | 14.07 | 10.22 | 4.88 | 2.53 | 1.74 | 1.41 | 1.27 | 1.23 | 1.16 |
| Oxolinic Acid | 0.89 | 1.33 | 1.63 | 1.77 | 2.02 | 1.86 | 0.96 | 1.06 | 1.05 | 1.14 | 1.17 |
| Nalidixic Acid | 1.50 | 1.76 | 1.74 | 0.87 | 0.68 | 0.91 | 1.18 | 1.25 | 1.26 | 1.26 | 1.22 |
| Erythromycin | 1.14 | 1.23 | 1.23 | 1.45 | 1.58 | 1.72 | 1.73 | 1.71 | 1.56 | 1.28 | 1.19 |
| Cephalexin | 1.73 | 1.78 | 1.76 | 1.62 | 1.68 | 1.81 | 1.11 | 1.11 | 1.12 | 1.11 | 1.12 |
| Penicillin G | 1.77 | 1.72 | 1.70 | 1.76 | 1.81 | 1.80 | 1.84 | 1.64 | 1.01 | 1.01 | 1.07 |
| Ampicillin | 1.76 | 1.73 | 1.77 | 1.76 | 1.72 | 1.75 | 1.85 | 1.81 | 1.16 | 1.02 | 1.06 |
| Vancomycin | 1.41 | 1.45 | 1.48 | 1.48 | 1.46 | 1.35 | 1.03 | 1.04 | 1.07 | 1.11 | 1.12 |
| Rifampicin | 14.00 | 13.91 | 14.12 | 14.47 | 15.30 | 8.95 | 3.16 | 1.87 | 1.37 | 1.22 | 1.20 |
| Spectinomycin | 1.30 | 1.23 | 1.21 | 1.14 | 1.16 | 1.14 | 1.08 | 1.10 | 1.10 | 1.10 | 1.13 |
| Bacitracin | 1.32 | 1.15 | 1.12 | 1.11 | 1.14 | 1.13 | 1.09 | 1.09 | 1.09 | 1.12 | 1.13 |
| DMSO | 1.14 | 1.14 | 1.14 | 1.14 | 1.14 | 1.14 | 1.14 | 1.14 | 1.14 | 1.14 | 1.14 |

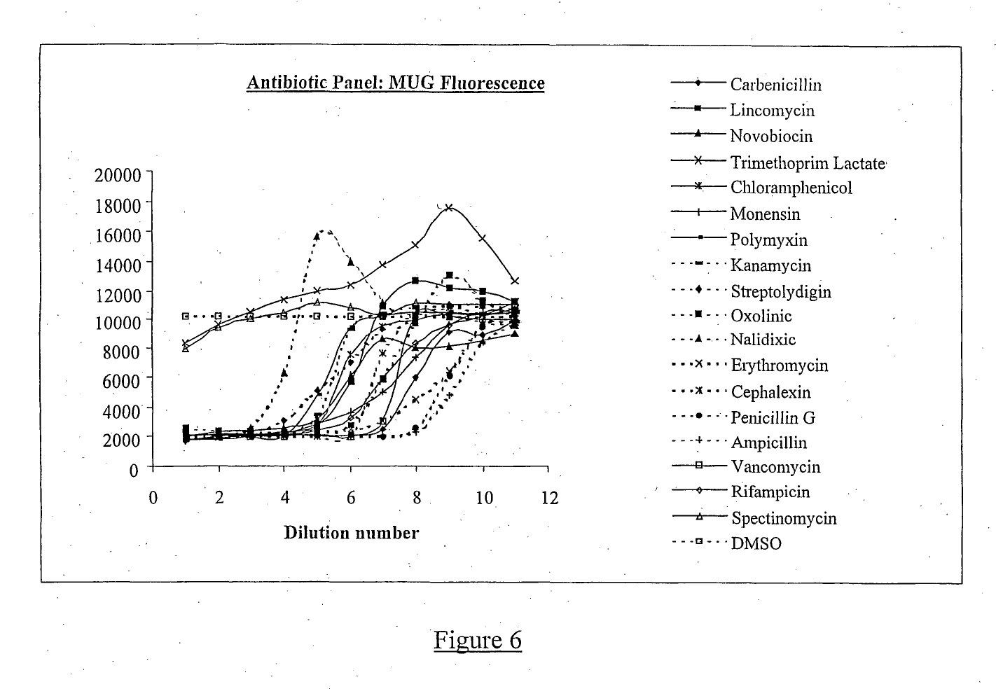

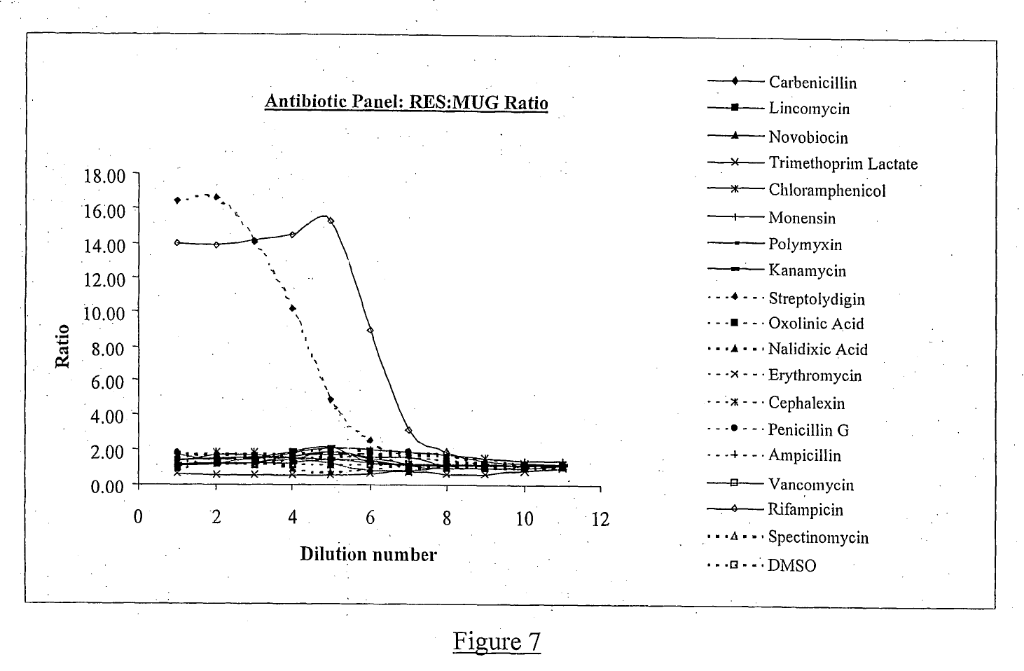

The results obtained in the experiment reveal that only two of the antibiotics tested are detected as specific inhibitors of RNA polymerase; rifampicin and streptolydigin. As shown in FIG. 5, the expression of the T7 RNAP-dependent control reporter is increased in their presence. In contrast, the test reporter, _{xyl}-lacZ, is reduced drastically in the presence of rifampicin or of streptolydigin (FIG. 6). Consequently, both of these antibiotics produce a RES:MUG ratio that is greater than that of the no antibiotic control (DMSO) at several concentrations (Table 2, FIG. 7). In the presence of all other antibiotics, except spectinomycin, both reporter activities were reduced (FIG. 5 and FIG. 6), though the drop in the control reporter activity was often less pronounced. Consequently, the ratio of control to test reporter expression (RES:MUG) for the majority of antibiotics is near to that for cells grown in the absence of antibiotics (DMSO only). Therefore, the assay could be used to distinguish between a compound specifically targeting bacterial RNAP and a non-specific inhibitor of B. subtilis growth. PL37 is resistant to spectinomycin and hence the reporter expression remained unchanged.

Discussion

A strain (PL37) that may be used in a screening assay for inhibitors of B. subtilis RNAP has been constructed. PL37 contains P_{xyl}-lacZ, a test reporter shown to monitor the activity of B. subtilis RNAP, and a control reporter, which is dependent on T7 RNAP for expression and independent of bacterial RNAP. The control reporter in PL37 contains the P_{T7} promoter separated by 9 bp from the xylose operator sequence, upstream of the gus gene (P_{T7X}-gus). PL37 was dependent on IPTG for expression of the control reporter, as the IPTG-inducible P_{spac} promoter regulates the gene encoding T7 RNAP. Experiments in the presence of a panel of antibiotics indicated that inhibitors of RNAP could be specifically detected in an assay measuring reporter activity of PL37. The antibiotic inhibitors of bacterial RNAP, rifampicin and streptolydigin, caused an increase in the ratio of control:test reporter activity compared to the ratio in the absence of any compound or in the presence of the non-specific antibiotic.

Sequence searches have indicated that the T7 RNAP is significantly similar only to RNAP of some other bacteriophages and to mitochondrial or chloroplast RNAP in certain plants and fungi. In the PL37-based dual reporter system, specific inhibitors of bacteriophage RNAP would be detected by the reduced expression of the P_{T7X}-gus reporter and the unchanged expression of the P_{xyl}-lacZ reporter, relative to those of the no compound control. Consequently, in the presence of such compounds the ratio of test to control reporter expression (MUG:RES) would be increased. This assay could therefore also be used to detect bacteriophage RNAP inhibitors.

### REFERENCES

(1) Studier, F. W. and Moffatt, B. A. (1986). Use of bacteriophage T7 RNA polymerase to direct selective high-level expression of cloned genes. J. Mol. Biol. 189:113-130.

(2) Karow, M. L. and Piggot, P. J. (1995) Construction of gusA transcriptional fusion vectors for Bacillus subtilis and their utilization for studies of spore formation. Gene 163: 69-74.

(3) Vagner, V. E. Dervyn and Ehrlich, S. D. (1998). A vector for systematic gene inactivation in Bacillus subtilis. Microbiology 144: 3097-3104.

(4) Stevens, C. M., Daniel, R. D., Illing, N. and Errington, J. (1992). Characterisation of a sporulation gene, spoIVA, involved in spore coat morphogenesis in Bacillus subtilis. J. Bacteriol. 174: 584-594.

(5) Sievers, J. (2000). Characterisation of the Bacillus subtilis genes ftsL and yyaA. D. Phil. thesis, University of Oxford.

(6) Daniel, R. D., Harry, E. J., Katis, V. L., Wale, R. G. and Errington, J. (1998). Characterisation of the essential cell division gene ftsL (yllD) of Bacillus subtilis and its role in the assembly of the division apparatus, Mol. Microbiol. 29: 593-604

1. A method for identifying a modulator of RNA polymerase (RNAP) comprising:
providing a host cell which expresses a first RNAP and having a first polynucleotide construct comprising a first promoter operably linked to a first gene, wherein the first gene is transcribed by the first RNAP; a second polynucleotide construct comprising a second promoter operably linked to a second gene which is a reporter gene, wherein the second reporter gene is transcribed by a second RNAP; and a source of the second RNAP;
contacting a test substance with the host cell under conditions that would permit the expression of the first and second genes in the absence of the test substance;
and determining thereby whether the said substance modulates RNAP.

2. A method according to claim 1 wherein the first gene comprises a reporter gene and the method comprises monitoring the expression of the first and second reporter genes.

3. A method according to claim 1 wherein the first gene is a repressor such as an unstable repressor of the second promoter such that expression of the first gene inhibits expression of the second reporter gene.

4. A method according to any one of the preceding claims wherein the second RNAP is derived from an heterologous organism to the first RNAP.

5. A method according to claim 4 wherein the source of heterologous RNAP comprises a third polynucleotide construct comprising a third promoter operably linked to a gene encoding heterologous RNAP.

6. A method according to claim 5 wherein the third promoter comprises a constitutive promoter.

7. A method according to claim 5 wherein the third promoter comprises an inducible promoter.

8. A method according to any one of the preceding claims wherein the second RNAP comprises a viral RNAP.

9. A method according to any one of claims 1 to 3 wherein the second RNAP comprises a second RNAP derived from the host cell.

10. A method according to any one of the preceding claims wherein the host cell comprises a bacterial cell.

11. A method according to claim 9 wherein the host cell comprises a bacterial cell and the first and second RNAP comprise σ^{A} and σ^{D/F}.

12. A method according to any one of the preceding claim wherein the first and/or second promoters are inducible promoters.

13. A method according to claim 12 wherein the first and second promoters are inducible in response to the stame stimulus.

14. A method according to any one of the preceding claims comprising determining whether the test substance modulates only one of the first or second RNAP activity.

15. A method according to claim 14 comprising determining whether a test substance inhibits the first RNAP but does not inhibit the second RNAP.

16. A method according to claim 15 wherein the host cell is a bacterium, comprising determining whether a test substance inhibits bacterial RNAP but does not inhibit the second RNAP.

17. An inhibitor of RNAP identifiable by the method of claim 15 or claim 16.

18. An inhibitor of RNAP identified according to the method of claim 15 or claim 16.

19. An inhibitor according to claim 17 or claim 18 for use in a method of treatment of a human or animal body.

20. An inhibitor according to claim 19 which inhibits bacterial RNAP for use as an antibiotic.

21. A pharmaceutical composition comprising the inhibitor of any one of claims 17, 18 or 19 and a pharmaceutically acceptable carrier.
